(19) United States
(12) Patent Application Publication
(10) Pub. No.: US 2005/0021036 A1
(43) Pub. Date: Jan. 27, 2005

(54) Self-drilling, self-tapping bone screw

(76) Inventors: Robin C. Whitmore, Simi Valley, CA (US); Todd Diamond, Simi Valley, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/624,735

(22) Filed: Jul. 21, 2003

(51) Int. Cl. A61B 17/56

(52) U.S. Cl. 606/73

(57) ABSTRACT

A self-drilling, self-tapping bone screw includes a body having a head at one end and a tip defining a generally flat cutting edge at an opposite end. A recess is formed in the head for receiving an end of an insertion tool. A dual lead thread extends radially outwardly from the body in a spiral path from the cutting tip towards the head. The dual lead thread pitch is tapered towards the cutting tip, and transitions to a straight thread towards the head.

### BACKGROUND OF THE INVENTION

The present invention generally relates to medical apparatuses, such as bone screws. More particularly, the present invention relates to a self-drilling, self-tapping bone screw having a dual lead thread.

In certain surgical procedures, such as repairing fractured bones, it is necessary to attach an item, such as a plate, to a bone. For example, in repairing fractures of the facial bones or of the cranial bones, it is common to use a thin metal bone plate to hold the various pieces together. In other systems, other fasteners are used.

To use such bone plates or fasteners, holes are drilled in the various bone pieces and the bone plate or fastener is then secured to the individual bones with bone screws. Disadvantageously, this requires two steps in order to insert the screw. First, the hole must be bored in the bone. Secondly, a self-tapping bone screw is screwed into the hole. While drilling a hole significantly reduces the torque the fastener experiences during insertion, there is a significant risk that fasteners inserted with this technique establish inadequate bone/screw contact to achieve adequate connection.

Although there exists supposedly self-drilling, self-tapping fasteners and screws, it has been found that such lack adequate strength to sustain the necessary torque in such applications, or still require drilling and tapping before inserting the screw into the cranial bone.

Accordingly, there remains a need for a bone screw which can be inserted without the need for drilling or tapping. There is also a need for a bone screw which is stabily inserted into the bone and which is self-drilling and self-tapping. What is further needed is a bone screw which has a very strong head to body connection so as to withstand the required torque for such insertion. The present invention fulfills these needs and provides other related advantages.

### SUMMARY OF THE INVENTION

The present invention resides in a self-drilling, self-tapping bone screw. The bone screw is comprised of a durable material, such as a medical grade titanium alloy. In a preferred embodiment, the bone screw is very small so as to be used in neurosurgery and craniofacial surgeries. As such, the bone screw is typically approximately 1.0-2.0 mm in diameter and approximately 3 to 6 mm in length.

The bone screw comprises a body having a head at one end, and a tip defining a generally flat cutting edge at an opposite end thereof. A recess is formed in the head which is configured to receive an end of an insertion tool, such as a driver bit or screw driver.

A dual lead thread extends radially outwardly from the body in a spiral path from the cutting tip edge towards the head. The dual lead thread is typically multi-pitched. In the preferred embodiment, the dual lead thread pitch is tapered towards the cutting tip and transitions to a straight thread towards the head.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of a bone screw embodying the present invention;

FIG. 2 is a cross-sectional view of the bone screw of the present invention taken generally along line 2-2 of FIG. 1;

FIG. 3 is an end view of the bone screw taken generally along line 3-3 of FIG. 1; and

FIG. 4 is an end view of the bone screw of the present invention taken generally along line 4-4 of FIG. 1.

### DETAILED DESCRIPTION OF THE PREFERRED EMBODIMENT

As shown in the accompanying drawings, for purposes of illustration, the present invention resides in a bone screw, generally referred to by the reference number 10. The bone screw 10 is designed such that it is self-drilling or self-boring, as well as self-tapping, thus eliminating the need for drilling and tapping to insert the screw into the bone, as with prior art screws. Although not limited to such, the bone screw 10 is primarily intended for use in neurosurgery and craniofacial surgery applications.

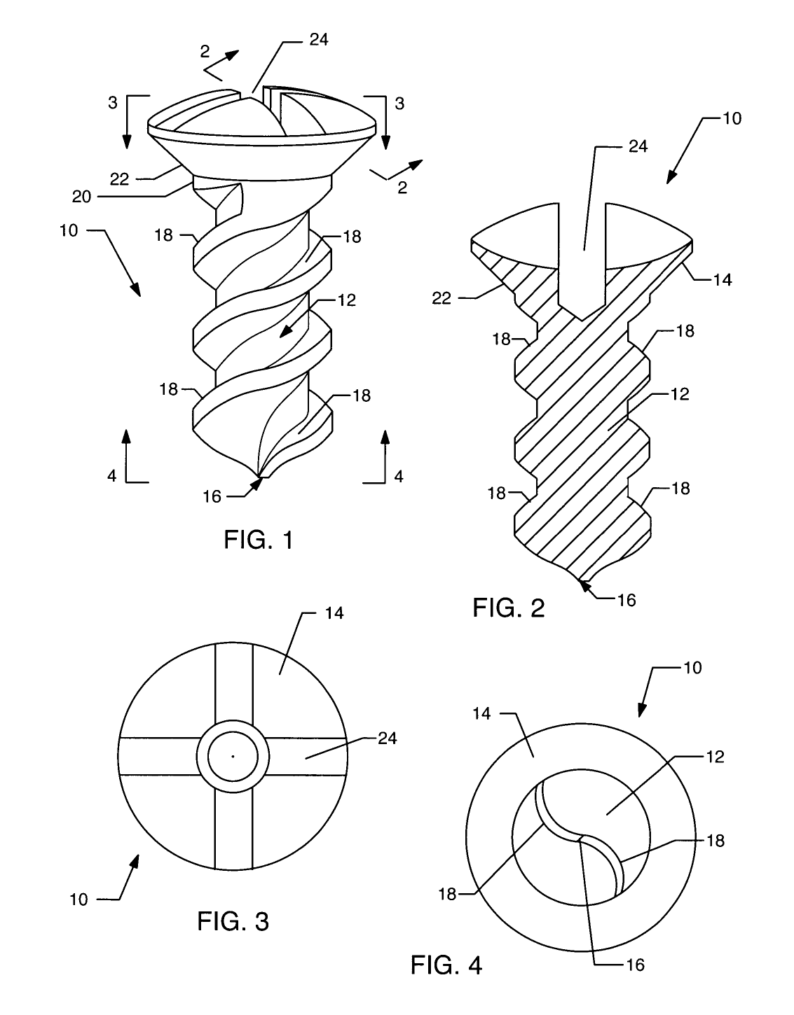

With reference now to FIGS. 1-4, the bone screw 10 is comprised of an elongated body or shank 12 portion having a head 14 at one end thereof and a cutting tip 16 at an opposite end thereof. In the preferred neurosurgery and craniofacial surgery usage, the screw 10 is approximately 1.0-2.0 mm in diameter, and approximately 3 to 6 mm in length. However, the dimensions can be altered to suit the needs of the particular application, and the invention is not intended to be limited to such dimensions for example, the diameter of the bone screw 10 of the present invention may be between 1.0 and 5.0 mm, and have a length of 3.0 to 100 mm for other medical procedures requiring bone screws of greater dimensions. The bone screw 10 is comprised of a hard and durable material, such as titanium which is medically acceptable for insertion into the body, and possesses sufficient strength to withstand the torque and tension exerted upon the screw 10 during installation. Preferably, the bone screw 10 is comprised of a medical grade titanium, such as TI6AL4V, or commercially pure titanium.

With particular reference to FIGS. 1 and 4, the cutting tip 16 is unique, in that it defines a generally flat cutting edge. The majority of screws are formed to a point. However, it has been found that the point promotes wobbling when the surgeon attempts to start the screw into the bone. It has been found that the flat cutting tip 16 provides a stable start into the bone. Also, the flat cutting edge 16 remains sharp, allowing for multiple removal and insertion of the same screw 10.

With reference now to FIGS. 1 and 2, a dual lead thread 18 and 18′ is formed on the body 12 so as to extend radially outwardly and form a spiral path from the cutting tip 16 towards the head 14. A double-lead or dual lead thread 18 and 18′ design provides an easy start of the screw 10 into the bone. It has also been found that such dual lead threads 18 and 18′ facilitate drilling into the bone, resulting in double the axial travel per turn. Thus, less turns are required to completely install the screw 10. Additionally, it has been found that the dual lead thread 18 and 18′ pulls bone chips out of the hole, whereas prior art bone screws can compress the bone chips inside of the hole.

The dual lead thread 18 and 18′ is multi-pitched. That is, the thread pitch is tapered towards the cutting tip 16, and transitions to a straight thread towards the head 14 of the screw 10. This allows an easier start of the screw 10 into the bone, and provides secure tightening with the bone. The cutting process for forming the dual thread 18 and 18′ design results in a 14.14 threads per inch on the cutting end, 29.32 threads per inch on the main body portion 12, and approximately 41.66 threads per inch of the pull-out portion of the screw 10. The multi-pitched thread design also provides superior strength at the head 14 to thread 18 or 18′ transition 20, as illustrated in FIG. 1. This provides higher strength due to less metal removal during the manufacturing process. Thus, the screw 10 of the present invention can withstand relatively high torque.

With reference now to FIGS. 1, 2 and 3, the head 14 has a generally frustro-conical shape with an angled bevel 22 extending between the body 12 and the head 14. The angle of the bevel 22 is optimized to prevent hole-through while minimizing the head's profile. A described above, the interface 20 between the head 14 and the body 12 is specifically designed to maximize the torque it can withstand and minimize the amount of material removed in the formation of the screw 10. The head 14 includes a recess 24 which is sized and shaped such so as to accept an end of an insertion tool. As illustrated, the recess 24 is in a form of a cruciform or slot so as to accept the end of a screw driver or driver bit. However, it should be understood that the recess 24 can comprise a slot, a hex-shaped recess, a square-shaped recess, etc. to accept the tips of different insertion tools.

The bone screw 10 of the present invention provides many advantages over comparable screws used previously. The flat cutting edge 16 promotes stability during insertion, and remains sharp so that the screw 10 can be removed and inserted multiple times. The dual-thread design 18 and 18′ enable the screw 10 to be self-drilling and self-tapping, saving time in the insertion process and providing a more secure and tight fit with the bone. A minimal amount of metal is removed during the manufacturing process so that the transition between the head 14 and the body 12 can withstand the high tension and torque exerted thereupon during the insertion and removal process.

Although an embodiment has been described in detail for purposes of illustration, various modifications may be made without departing from the scope as part of the invention. Accordingly, the invention is not to be limited, except as by the appended claims.

1. A self-drilling bone screw, comprising:
a body having a head at one end and a tip defining a generally flat cutting edge at an opposite end thereof; and
a dual lead thread extending radially outwardly from the body in a spiral path from the cutting tip towards the head.

2. The bone screw of claim 1, wherein the dual lead thread is multi-pitched.

3. The bone screw of claim 2, wherein the dual lead thread pitch is tapered towards the cutting tip and transitions to a straight thread towards the head.

4. The bone screw of claim 1, including a recess formed in the head configured to receive an end of an insertion tool.

5. The bone screw of claim 1, wherein the bone screw is comprised of a medical grade titanium alloy.

6. The bone screw of claim 1, wherein the bone screw is approximately 1.0 to 2.0 mm in diameter and approximately 3.0 to 6.0 mm in length.

7. A self-drilling bone screw, comprising:
a body having a head at one end and a tip defining a generally flat cutting edge at an opposite end thereof; and
a dual lead thread extending radially outwardly from the body in a spiral path from the cutting tip towards the head, the dual lead thread being multi-pitched such that the pitch of the thread is tapered towards the cutting tip and transitions to a straight thread towards the head.

8. The bone screw of claim 7, including a recess formed in the head configured to receive an end of an insertion tool.

9. The bone screw of claim 7, wherein the bone screw is comprised of a medical grade titanium alloy.

10. The bone screw of claim 7, wherein the bone screw is approximately 1.0 to 2.0 mm in diameter and approximately 3.0 to 6.0 mm in length.

11. A self-drilling, self-tapping bone screw, comprising:
a body comprised of medical grade titanium alloy of approximately 1.0 to 2.0 mm in diameter and approximately 3.0 to 6.0 mm in length, the body having a head at one end and a tip defining a generally flat cutting edge at an opposite end thereof;
a dual lead thread extending radially outwardly from the body in a spiral path from the cutting tip towards the head, the dual lead thread being multi-pitched such that the pitch of the thread is tapered towards the cutting tip and transitions to a straight thread towards the head; and
a recess formed in the head configured to receive an end of an insertion tool.
